(19) United States
(12) Patent Application Publication
(10) Pub. No.: US 2021/0318319 A1
(43) Pub. Date: Oct. 14, 2021

(54) DETECTION OF BIOMARKERS

(71) Applicant: Imperial College of Science, Technology and Medicine, London (GB)

(72) Inventors: George Hanna, London (GB); Piers Boshier, London (GB); Ilaria Belluomo, London (GB)

(21) Appl. No.: 16/766,956

(22) PCT Filed: Nov. 26, 2018
(86) PCT No.: PCT/GB2018/053407

§ 371 Date: May 26, 2020

(30) Foreign Application Priority Data
Nov. 27, 2017 (GB) 1719639.5

(51) Int. Cl. G01N 33/574; G01N 33/497

(52) U.S. Cl. CPC G01N 33/57488; G01N 33/57446; G01N 33/497; G01N 2333/245; G01N 2033/4977; G01N 2333/315; G01N 2333/33; G01N 2333/335; G01N 2333/26

(57) ABSTRACT

The invention relates to the detection of biomarkers, and methods, compositions and kits for the detection of such biological markers for diagnosing various conditions, such as cancer. In particular, the invention relates to the detection of compounds as diagnostic and prognostic markers for detecting cancer, such as oesophago-gastric cancer.

The present invention relates to the detection of biomarkers, and particularly although not exclusively, to methods, compositions and kits for the detection of biological markers for diagnosing various conditions, such as cancer. In particular, the invention relates to the detection of compounds as diagnostic and prognostic markers for detecting cancer, such as oesophago-gastric cancer.

Oesophageal adenocarcinoma is among the most common five cancers and has the fastest rising incidence of any cancer in the Western population. The UK has the highest incidence of oesophageal adenocarcinoma worldwide. Stomach cancer is the third leading cause of cancer death worldwide. Five-year survival for oesophageal and gastric cancer in the UK remains very poor (13% and 18% respectively), among the worst in Europe. The key to improving cancer-survival is earlier diagnosis. However, symptoms are non-specific and commonly-shared with benign diseases. By the time symptoms become cancer-specific, the disease is often at an advanced stage with poor prognosis. Cancer burden and unnecessary investigations of patients with non-specific symptoms result in substantial costs. There is, thus, an urgent need for a non-invasive test for patients with non-specific gastrointestinal symptoms in order to effectively triage patients to have endoscopy and other diagnostic modalities.

Prior research has shown an association between oesophago-gastric cancer and volatile organic compounds (VOCs), and an approach for its diagnosis is exhaled breath testing. Researchers using gas chromatography mass spectrometry (GC-MS) have suggested the existence of a breath volatile organic compounds (VOCs) profile specific to a specific cancer [4]. GC-MS is a good technique for VOC identification, but it is semi-quantitative in nature, unless robust calibration curves employed, and therefore limited in its ability of research findings to be reproduced by different research groups. Furthermore, there is a substantial analytical time for each sample, which does not naturally lend itself to high throughput analysis. Direct injection mass spectrometry, such as selected ion flow tube mass spectrometry (SIFT-MS) and proton transfer reaction time of flight mass spectrometry (PTR-ToF-MS) have the advantage of being quantitative and permit real-time analysis [5,6].

What is required is a reliable non-invasive diagnostic test to identify patients suffering from cancers, such as oesophago-gastric cancer. A diagnostic method to identify those patients with cancer would be of immense benefit to patients and would raise the possibility of early treatment and improved prognosis.

The inventors have developed a non-invasive test for cancer based on the detection of signature compounds, such as volatile organic compounds (VOCs), in exhaled breath. Improved accuracy of this test is achieved by means of administering an oral stimulus foodstuff (e.g. a drink, capsule or solid foodstuff), which transiently induces or “stimulates” cancer and its associated microbiome to produce greater quantities of distinctive signature compounds (e.g. VOCs), and thereby improving test performance io and diagnostic accuracy. This will allow patients with non-specific symptoms, yet at a high-risk of oesophago-gastric cancer, to be identified earlier and referred for further investigation and treatment.

In a first aspect of the invention, there is provided a method for diagnosing a subject suffering from cancer, or a pre-disposition thereto, or for providing a prognosis of the subject's condition, the method comprising:
(i) detecting, in a bodily sample from a test subject, the concentration of a signature compound resulting from the metabolism by a cancer-associated microorganism, of at least one substrate in a composition previously administered to the subject; and
(ii) comparing this concentration with a reference for the concentration of the signature compound in an individual who does not suffer from cancer, wherein an increase or a decrease in the concentration of the signature compound compared to the reference, suggests that the subject is suffering from cancer, or has a pre-disposition thereto, or provides a negative prognosis of the subject's condition.

In a second aspect, there is provided a method for detecting a signature compound in a test subject, the method comprising:

(i) providing the subject with a composition comprising at least one substrate which is suitable for metabolism by cancer-associated microorganism into a signature compound; and (ii) detecting the concentration of the signature compound in a bodily sample from the subject.

In a third aspect, there is provided a composition comprising at least one substrate which is suitable for metabolism by a cancer-associated microorganism into a signature compound, for use in a method of diagnosis or prognosis, preferably of cancer.

In a fourth aspect, there is provided a composition comprising at least one substrate which is suitable for metabolism by a cancer-associated microorganism into a signature compound, for use in the method of the first or the second aspect.

In a fifth aspect, there is provided a kit for diagnosing a subject suffering from cancer, or a pre-disposition thereto, or for providing a prognosis of the subject's condition, the kit comprising:

(a) a composition comprising at least one substrate which is suitable for metabolism by cancer-associated microorganism into a signature compound;

(b) means for determining the concentration of a signature compound in a sample from is a test subject; and

(c) a reference for the concentration of the signature compound in a sample from an individual who does not suffer from cancer,

wherein the kit is used to identify an increase or a decrease in the concentration of the signature compound in the bodily sample from the test subject, compared to the reference, thereby suggesting that the subject suffers from cancer, or has a pre-disposition thereto, or provides a negative prognosis of the subject's condition.

Methods of the first and second aspect may comprise administering or having administered, to the subject, a therapeutic agent or putting the subject on a specialised diet, wherein the therapeutic agent or the specialised diet prevents, reduces or delays progression of cancer.

Thus, in a sixth aspect, there is provided a method of treating a subject suffering from cancer, said method comprising the steps of:
(i) providing the subject with a composition comprising at least one substrate which is suitable for metabolism by a cancer-associated microorganism into a signature compound;
(ii) analysing the concentration of the signature compound in a bodily sample from a test subject and comparing this concentration with a reference for the concentration of the signature compound in an individual who does not suffer from cancer, wherein an increase or a decrease in the concentration of the signature compound in the bodily sample from the test subject compared to the reference suggests that the subject is suffering from cancer, or has a pre-disposition thereto, or has a negative prognosis; and
(iii) administering or having administered, to the subject, a therapeutic agent or putting the subject on a specialised diet, wherein the therapeutic agent or the specialised diet prevents, reduces or delays progression of cancer.

In a seventh aspect, there is provided a method for determining the efficacy of treating a subject suffering from cancer with a therapeutic agent or a specialised diet, io the method comprising:
(i) providing the subject with a composition comprising at least one substrate which is suitable for metabolism by a cancer-associated microorganism into a signature compound; and
(ii) analysing the concentration of the signature compound in a bodily sample from a test subject, and comparing this concentration with a reference for the concentration of the signature compound in an individual who does not suffer from cancer,
wherein an increase or a decrease in the concentration of the signature compound in the bodily sample from the test subject compared to the reference suggests that the treatment regime with the therapeutic agent or the specialised diet is effective or ineffective.

Advantageously, the benefits for patients with cancer are earlier detection in a population who may have vague or undetectable symptoms. The diagnostic or prognostic methods of the invention can be offered immediately by a medical professional in a similar manner to a routine blood test, thus avoiding the need to “watch-and-wait” to see if a patient's symptoms worsen. Earlier detection significantly improves survival rates. It will be appreciated that “diagnosis” can mean the identification of the nature of an illness or condition, and that “prognosis” can mean predicting the rate of progression or improvement and/or duration of the condition. A prognosis method may be performed subsequent to, and separately from, an initial diagnosis.

Furthermore, the methods could increase the proportion of appropriate referrals from primary care to have endoscopy and could potentially improve referral guidelines. The methods will also provide the opportunity to test younger patients than NICE-age threshold for referral. Patients without cancer will avoid invasive tests and anxiety while awaiting endoscopy. Avoiding unnecessary investigations would free up resources that could be used to save lives. Enhancing the diagnostic pathway will improve patient experience.

Preferably, the methods of the invention comprise an initial step of providing the subject with the composition comprising the at least one substrate which is suitable for metabolism by a cancer-associated microorganism into a signature compound.

Preferably, the methods of the invention comprise analysing the concentration of the signature compound in the bodily sample from the test subject and comparing this concentration with the reference for the concentration of the signature compound in an individual who does not suffer from cancer.

In an embodiment, the cancer-associated microorganism is associated with oesophago-gastric junction cancer, gastric cancer, oesophageal cancer, oesophageal squamous-cell carcinoma (ESCC), or oesophageal adenocarcinoma (EAC). Therefore, in a preferred embodiment, the diagnosis is for diagnosing oesophago-gastric junction cancer, gastric cancer, oesophageal cancer, oesophageal squamous-cell carcinoma (ESCC), or oesophageal adenocarcinoma (EAC). Most preferably, the micro-organism is associated with oesophago-gastric cancer, such that this condition can be diagnosed or prognosed.

In an embodiment, the cancer-associated microorganism is associated with pancreatic cancer or colorectal cancer. Accordingly, the diagnosis may be for diagnosing pancreatic cancer or colorectal cancer.

The methods of the invention are useful for monitoring the efficacy of a putative treatment for the relevant cancer. For example, the treatment for resectable oesophago-gastric cancer may comprise neoadjuvant chemotherapy, or chemoradiotherapy followed by surgery and adjuvant chemotherapy. The treatment for very early stage oesophago-gastric cancer may comprise endoscopic resection. The treatment for advanced oesophago-gastric cancer may comprise palliative chemotherapy. It has recently been shown that cancer-associated microbiome enhances metastasis to the liver (Bullman et al., Science, 2017). Hence, the invention described herein may be used to monitor the response of therapy directed towards the cancer-associated microbiome.

If the cancer is pancreatic cancer, then treatment may comprise administration of chemotherapy, chemoradiotherapy with or without surgery. For example, if the cancer is colorectal cancer, then treatment may comprise administration of chemotherapy, chemoradiotherapy with or without surgery, or endoscopic resection.

The composition comprising at least one substrate, which is suitable for metabolism by a cancer-associated microorganism into a signature compound, is ingested by the subject. The composition may be solid or fluid, which may be eaten or swallowed. In an embodiment, the composition may be chewable, which results in release of the substrate and it being taken down into the gut. In an embodiment, the composition comprising the at least one substrate may be in the form of a capsule that is designed to degrade at a certain position with the gastrointestinal tract, thereby offering targeted release of the at least one substrate. However, the composition is preferably a liquid (i.e. a drink), which may be swallowed, and which may be referred to as an oral stimulant drink (OSD).

The at least one substrate may be any molecule or compound that can be metabolised by a cancer-associated microorganism, either directly or indirectly, into a signature compound. The at least one substrate may be selected from a group consisting of: tyrosine, acetic acid, ethanol, lactic acid, lactate, glutamic acid, glutamate, glycerol, D-glucose, D-sucrose, D-lactose, D-fructose, D-mannose, D-gulose, D-galactose, D-Xylose, D-arabinose, D-lyxose, D-ribose, L-arabinose, L-rhamnose, L-xylulose, di-, trioligo and poly-saccharides, sorbitol, c₄, c₇ and >c8 monosaccharides, pyruvic acid, ascorbic acid, malic acid, citric acid, succinic acid, fumaric acid, oxalic acid, tannic acid, tartaric acid, sorbitol, mannitol, maltitol, lactitol, erythritol, palmitic acid, stearic acid, oleic acid, linoleic acid, arachidonic acid, triglycerides, glycolipids, any or all of the 20 proteinogenic amino acids, 2-amino butyric acid, ornithine, canavanine, homoarginine, artificial sweeteners (e.g. stevia, aspartame, sucralose), preservatives (e.g. E numbers, nitrate), and small molecule inducers.

The composition may comprise any combination of the aforementioned substrates. Preferably, the composition comprises or consists one or more substrate selected from the group consisting of: glucose, sorbitol, lactose tyrosine, glutamic acid, glycerol, citric acid and acetic acid, or any combination thereof.

Preferably, the composition comprises glucose, preferably at a concentration of between about 7000 mg/100 mL and 20000 mg/100 mL, more preferably between 9000 mg/100 mL and 17000 mg/100 mL, and most preferably between 11000mg/100 mL and 15000 mg/100 mL.

Preferably, the composition comprises lactose, preferably at a concentration of between about 7000 mg/100 mL and 20000 mg/100 mL, more preferably between 9000 mg/100 mL and 17000 mg/100 mL, and most preferably between n11000 mg/100 mL and 15000 mg/100 mL.

Preferably, the composition comprises sorbitol, preferably at a concentration of between about 1000 mg/100 mL and 6000 mg/100 mL, more preferably between 2000 mg/100 mL and 5000 mg/100 mL, and most preferably between 3000 mg/100 mL and 4000 mg/100 mL.

Preferably, the composition comprises tyrosine, preferably at a concentration of between about 25 mg/100 mL and 500 mg/100 mL, more preferably between 50 mg/100 mL and 400 mg/100 mL, and most preferably between 100 mg/100 mL and 300 mg/100 mL.

Preferably, the composition comprises glutamic acid, preferably at a concentration of between about 500 mg/100 mL and 5000 mg/100 mL, more preferably between 100 mg/100 mL and 3500 mg/100 mL, and most preferably between 1500 mg/100 mL and 2500 mg/100 mL.

Preferably, the composition comprises glycerol, preferably at a concentration of between about 1000 mg/100 mL and 30000 mg/100 mL, more preferably between 14000 mg/100 mL and 25000 mg/100 mL, and most preferably between 17000 mg/100 mL and 22000 mg/100 mL.

Preferably, the composition comprises citric acid, preferably at a concentration of between about 500 mg/100 mL and 3000 mg/100 mL, more preferably between 1000 mg/100 mL and 2000 mg/100 mL, and most preferably between 1200 mg/100 mL and 1700 mg/100 mL.

Preferably, the composition comprises acetic acid, preferably at a concentration of between about 200 mg/100 mL and 1500 mg/100 mL, more preferably between 400 mg/100 mL and 100 mg/100 mL, and most preferably between 600 mg/100 mL and 800 mg/100 mL.

Preferably, the composition comprises or consists of at least two, three or four of the substrates selected from the group consisting of tyrosine, glutamic acid, glucose, sorbitol, lactose, glycerol, citric acid and acetic acid. Preferably, the composition comprises or consists of at least five, six, seven or eight of the substrates selected from io the group consisting of tyrosine, glutamic acid, glucose, sorbitol, lactose, glycerol, citric acid and acetic acid.

The inventors believe that glucose, lactose and sorbitol are especially useful as substrate in the composition for metabolism by a cancer-associated microorganism into the signature compound. Most preferably, therefore, the composition comprises at least one substrate selected from glucose, lactose and sorbitol. It will be appreciated, however, that tyrosine, glutamic acid, glycerol, citric acid and/or acetic acid may also be included in the composition in any of the above concentrations.

The composition may be an existing composition, foodstuff or drink, which comprises any one of the aforementioned constituents.

The cancer-associated microorganism may be a bacterium. It will be appreciated that the microorganisms and bacteria present in the gut form the so-called “microbiome”. Therefore, the cancer-associated microorganism that metabolises the at least one substrate into a signature compound, which is detected and/or analysed in the methods of the invention to diagnose cancer, preferably form part of the microbiome.

The cancer-associated microorganism may be Streptococcus, Lactobacillus, Veillonella, Prevotella, Neisseria, Haemophilus, L. coleohominis, Lachnospiraceae, Klebsiella, Clostridiales, Erysipelotrichales, or any combination thereof.

The cancer-associated microorganism may be S. pyogenes, Klebsiella pneumoniae, Lactobacillus acidophilus, or any combination thereof.

The cancer-associated microorganism may be E. coli, P. mirabili, B. cepacia, S. pyogenes, Streptococcus salivarius, Actinomyces naeslundii, Lactobacillus fermentum, Streptococcus anginosus, Clostridium bifermentans, Clostridium perfringens, Clostridium septicum, Clostridium sporogenes, Clostridium tertium, Eubacterium lentum, Eubacterium sp., Fusobacterium simiae, Fusobacterium necrophorum, Lactobacillus acidophilus, Peptococcus niger, Peptostreptococcus anaerobius, Peptostreptococcus asaccharolyticus, Peptostreptococcus prevotii, P. aeruginosa, S. aureus, P. mirabilis, E. faecalis, S. pneumoniae, N. meningitides, Acinetobacter baumannii, Bacteroides capillosus, Bacteroides fragilis, Bacteroides pyogenes, Clostridium difficile, Clostridium ramosum, Enterobacter cloacae, Klebsiella pneumoniae, Nocardiasp., Propionibacterium acnes, Propionibacterium propionicum, or any combination thereof. Preferably, the cancer-associated microorganism is E. coli, L. fermentum, S. salivarius, S. anginosus or K. pneumoniae.

The subject may be any animal of veterinary interest, for instance, a cat, dog, horse etc. However, it is preferred that the subject is a mammal, such as a human, either male or female.

Preferably, a sample is taken from the subject, and the signature compound in the bodily sample is then detected. In some embodiments, the concentration of the signature compound is measured.

A signature compound may be any compound that can indicate or correlate with the presence of a microorganism. The signature compounds, which are detected, may be volatile organic compounds (VOCs), which lead to a fermentation profile, and they may be detected in the bodily sample by a variety of techniques. In one embodiment, these compounds may be detected within a liquid or semi-solid sample in which they are dissolved. In a preferred embodiment, however, the compounds are detected from gases or vapours. For example, as the signature compounds are VOCs, they may emanate from, or form part of, the sample, and may thus be detected in gaseous or vapour form.

Preferably, the volatile organic compound (VOC) is selected from a group consisting of: butyric acid, gamma amino butyric acid, caproic acid, hydrogen sulphide, pentanol, propanoic acid, acetic acid, 1,2-propanediol, ethanol, and 3-hydroxypropionic acid, or any combination thereof.

The VOCs may be aldehydes, fatty acids, alcohols, or any combination thereof.

The VOCs may be a C₁-C₃ aldehyde, a C₁-C₃ alcohol, a C₂-C₁₀ alkane wherein a first carbon atom is substituted with the ═O group and a second carbon atom is substituted with an —OH group, a C₁-C₂₀ alkane, a C₄-C₁₀ alcohol, a C₁-C₆ carboxylic acid, a C₄-C₂₀ aldehyde, a C₂ aldehyde, a C₃ aldehyde, a C₈ aldehyde, a C₉ aldehyde, a C₁₀ aldehyde, a C₁₁ aldehyde, an analogue or derivative of any aforementioned species, or any combination thereof. The VOCs may be propanal, nonanal, decanal, formaldehyde, methanol, pentane, isopropyl alcohol, n-hexane, 1-butanol, acetoin, propanoic acid, undecanal, tetradecane, or any combination thereof. In another embodiment, the VOCs may be acetone, acetic acid, butyric acid, pentanoic acid, hexanoic acid, phenol, ethyl phenol, acetaldehyde, or any combination thereof. In yet another embodiment, the VOCs may be hexanoic acid, pentanoic acid, acetic acid, 2 ethyl phenol, or any combination thereof.

In one embodiment, a composition comprising acetic acid and/or ethanol (i.e. the substrate which is metabolised by the cancer-associated microorganism) may be provided to a subject, in order to increase the concentration of the signature compound, butyric acid and/or caproic acid. The concentrations of these signature compounds may then be analysed in order to indicate the presence of Clostridium spp. and hence provide a diagnosis, indicate a pre-disposition thereto, or provide a prognosis of, for instance, oesophageal squamous-cell carcinoma. Evidence for the association is shown in Example 3 below. Hence, preferably the method is used to provide a diagnosis, indicate a pre-disposition thereto, or provide a prognosis of, oesophageal squamous-cell carcinoma, wherein the composition comprises a substrate selected from acetic acid and/or ethanol, which is preferably metabolised to a signature compound selected from butyric acid and/or caproic acid, which is preferably analysed to indicate the presence of Clostridium spp.

In another embodiment, a composition comprising lactic acid (i.e. the substrate) may be provided to a subject, in order to increase the concentration of the signature compound, acetic acid, 1,2-propanediol, and/or ethanol. The concentration of these signature compounds may then be analysed in order to indicate the presence of Lactobacillus spp. and hence provide a diagnosis, indicate a pre-disposition thereto, or provide a prognosis of, for instance, gastric cancer. Evidence for the association is shown in Example 3 below. Accordingly, preferably the method is used to provide a diagnosis, indicate a pre-disposition thereto, or provide a prognosis of, gastric cancer, wherein the composition comprises a substrate which is lactic acid, which is preferably metabolised to a signature compound selected from acetic acid, 1,2-propanediol, and/or ethanol, which is preferably analysed to indicate the presence of Lactobacillus spp.

In yet another embodiment, a composition comprising glutamate (i.e. the substrate) may be provided to a subject, in order to increase the concentration of the signature compound, gamma amino butyric acid. The concentration of this signature compound may then be analysed in order to indicate the presence of Lactococcus spp., Clostridium spp., and others, and hence provide a diagnosis, indicate a pre-disposition thereto, or provide a prognosis of oesophago-gastric cancer. Evidence for the association is shown in Example ₃ below. Thus, preferably the method is used to provide a diagnosis, indicate a pre-disposition thereto, or provide a prognosis of, oesophago-gastric cancer, wherein the composition comprises a substrate which is glutamate, which is preferably metabolised to a signature compound which is gamma amino butyric acid, which is preferably analysed to indicate the presence of Lactococcus spp. or Clostridium spp.

In still another embodiment, a composition comprising glycerol (i.e. the substrate) may be provided to a subject, in order to increase the concentration of the signature compound, 3-hydroxypropionic acid. The concentration of this signature compound may then be analysed in order to indicate the presence of Klebsiella spp. and hence provide a diagnosis, indicate a pre-disposition thereto, or provide a prognosis of gastric cancer. Evidence for the association is shown in Example 3 below. Therefore, preferably the method is used to provide a diagnosis, indicate a pre-disposition thereto, or provide a prognosis of, gastric cancer, wherein the composition comprises a substrate which is glycerol, which is preferably metabolised to a signature compound which is 3-hydroxypropionic acid, which is preferably analysed to indicate the presence of Klebsiella spp.

The kit of the fifth aspect may comprise sample extraction means for obtaining the sample from the test subject. The sample extraction means may comprise a needle or syringe or the like. The kit may comprise a sample collection container for receiving the extracted sample, which may be liquid, gaseous or semi-solid. The kit may further comprise instructions for use.

Preferably, the sample is any bodily sample into which the signature compound is present or secreted. Preferably, the detection or diagnostic method is therefore performed in vitro. For example, the sample may comprise urine, faeces, hair, sweat, saliva, blood, or tears. In one embodiment, the sample may be assayed for the signature compound's levels immediately. Alternatively, the sample may be stored at low temperatures, for example in a fridge or even frozen before the concentration of signature compound is determined. Measurement of the signature compound in the bodily sample may be made on the whole sample or a processed sample, for instance whole blood or processed blood.

In an embodiment, the sample may be a urine sample. It is preferred that the is concentration of the signature compound in the bodily sample is measured in vitro from a urine sample taken from the subject. The compound may be detected from gases or vapours emanating from the urine sample. It will be appreciated that detection of the compound in the gas phase emitted from urine is preferred.

It will also be appreciated that “fresh” bodily samples may be analysed immediately after they have been taken from a subject. Alternatively, the samples may be frozen and stored. The sample may then be de-frosted and analysed at a later date.

Most preferably, however, the bodily sample may be a breath sample from the test subject. The sample may be collected by the subject performing exhalation through the mouth, preferably after nasal inhalation. Preferably, the sample comprises the subject's alveolar air. Preferably, the alveolar air is collected over dead space air by capturing end-expiratory breath. VOCs from breath bags are then preferably pre-concentrated onto thermal desorption tubes by transferring breath across the tubes.

The difference in concentration of signature compound, which would indicate cancer in the subject or a predisposition thereto, may be an increase or a decrease compared to the reference. It will be appreciated that the concentration of signature compound in patients suffering from a disease is highly dependent on a number of factors, for example how far the disease has progressed, and the age and gender of the subject. It will also be appreciated that the reference concentration of signature compound in individuals who do not suffer from the disease may fluctuate to some degree, but that on average over a given period of time, the concentration tends to be substantially constant. In addition, it should be appreciated that the concentration of signature compound in one group of individuals who suffer from a disease may be different to the concentration of that compound in another group of individuals who do not suffer from the disease. However, it is possible to determine the average concentration of signature compound in individuals who do not suffer from the cancer, and this is referred to as the reference or ‘normal’ concentration of signature compound. The normal concentration corresponds to the reference values discussed above.

In one embodiment, the methods of the invention preferably comprise determining the ratio of chemicals within the breath (i.e. use other components within it as a reference), and then compare these markers to the disease to show if they are elevated or reduced.

The signature compound is preferably a volatile organic compound (VOC), which provides a profile, and it may be detected in or from the bodily sample by a variety of techniques. Thus, these compounds may be detected using a gas analyser. Examples of suitable detector for detecting the signature compound preferably includes an electrochemical sensor, a semiconducting metal oxide sensor, a quartz crystal microbalance sensor, an optical dye sensor, a fluorescence sensor, a conducting polymer sensor, a composite polymer sensor, or optical spectrometry.

The inventors have demonstrated that the signature compounds can be reliably detected using gas chromatography, mass spectrometry, GCMS or TOF. Dedicated sensors could be used for the detection step.

The reference values may be obtained by assaying a statistically significant number of control samples (i.e. samples from subjects who do not suffer from the disease). Accordingly, the reference (ii) according to the kit of the fifth aspect of the invention may be a control sample (for assaying).

The apparatus preferably comprises a positive control (most preferably provided in a container), which corresponds to the signature compound(s). The apparatus preferably comprises a negative control (preferably provided in a container). In a preferred embodiment, the kit may comprise the reference, a positive control and a negative control. The kit may also comprise further controls, as necessary, such as “spike-in” controls to provide a reference for concentration, and further positive controls for each of the signature compounds, or an analogue or derivative thereof.

Accordingly, the inventors have realised that the difference in concentrations of the signature compound between the reference normal (i.e. control) and increased/decreased levels, can be used as a physiological marker, suggestive of the presence of a disease in the test subject. It will be appreciated that if a subject has an increased/decrease concentration of one or more signature compounds which is considerably higher/lower than the ‘normal’ concentration of that compound in the reference, control value, then they would be at a higher risk of having the disease, or a condition that was more advanced, than if the concentration of that compound was only marginally higher/lower than the ‘normal’ concentration.

The skilled technician will appreciate how to measure the concentrations of the signature compound in a statistically significant number of control individuals, and the concentration of compound in the test subject, and then use these respective figures to determine whether the test subject has a statistically significant increase/decrease in the compound's concentration, and therefore infer whether that subject is suffering from the disease for which the subject has been screened.

All features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figure, in which:

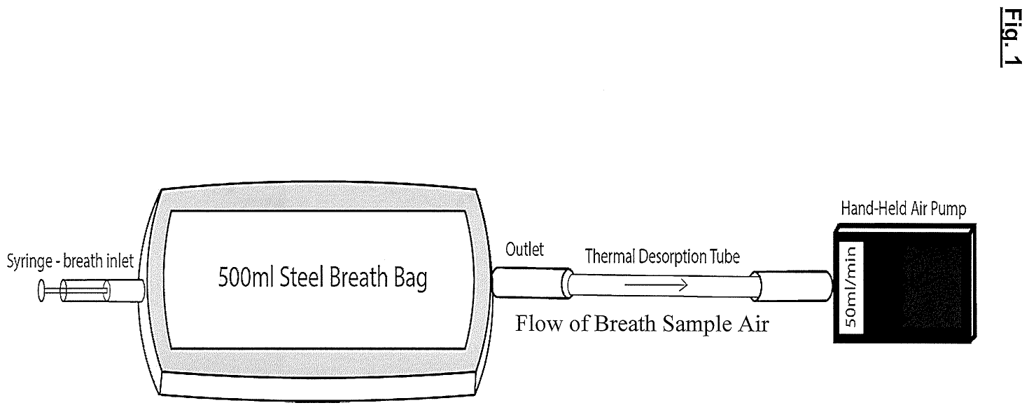

FIG. 1 shows an embodiment of an apparatus and a method used for concentrating VOCs from steel breath bags onto thermal desorption tubes;

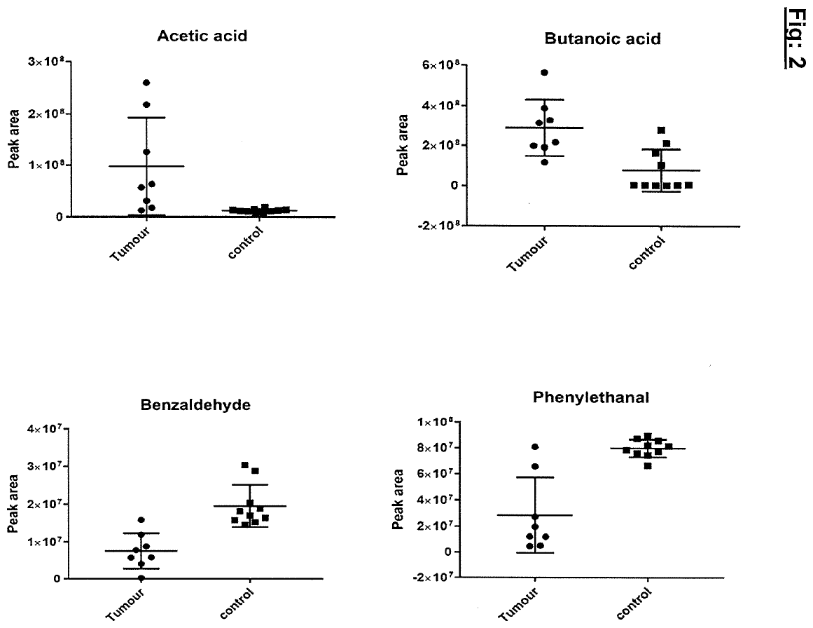

FIG. 2 shows VOC production by overall species in patient cancer tissues compared to non-cancer control;

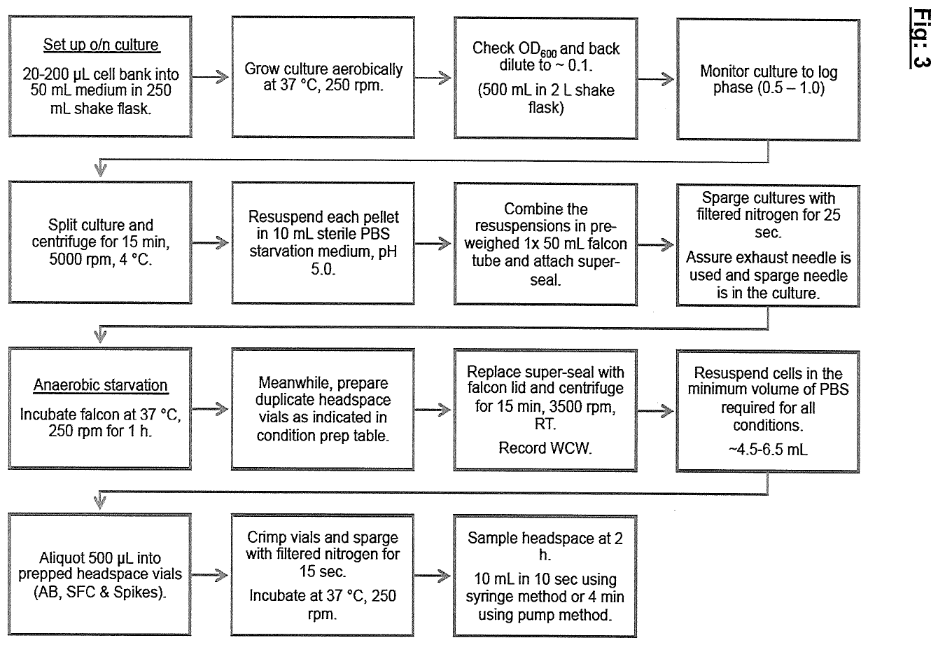

FIG. 3 shows high throughput in vitro culture stimulation and VOC sampling protocol; and

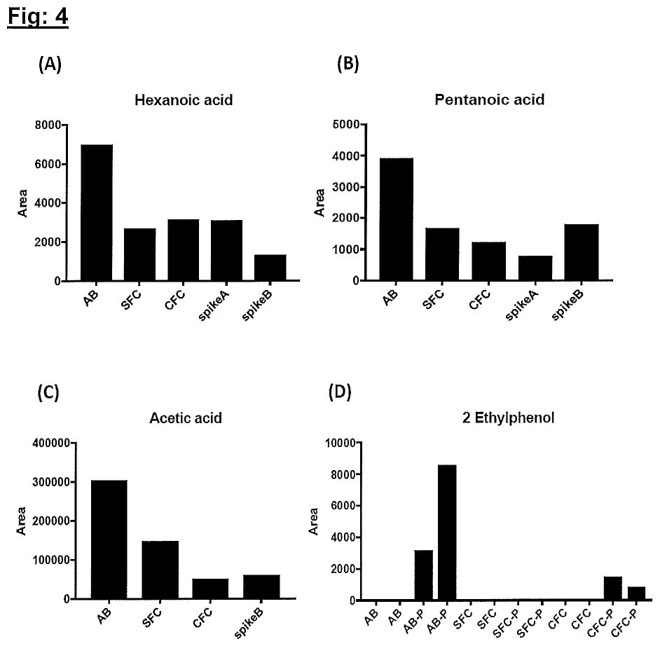

FIG. 4 shows examples of elevated VOCs in the headspace of (A) Escherichia coli in glucose media, (B) Klebsiella pneumonia in glucose media, (C) Streptococcus salivarius in glycerol media, and (D) Lactobacillus fermentum in glycerol media. Active biotransformation (AB) compared to controls comprising bacterial cultures that were stimulant-free (SFC) and stimulant compositions that were free of bacterial cultures (CFC). Spike A and B contained 3-ethyl phenol and hexanoic acid as internal compound standards as quality measures. Samples with −P denotes a vacuum pump was used to sample the headspace of the biotransformation vessel into thermal desorption tubes. Data is derived from VOC analysis by GC-MS.

### EXAMPLES

### Example 1

### Microbial VOCs Produced in Response to Particular Stimulants found in Food

Table 1 shows VOCs that can be detected to indicate the presence of specific bacteria, and the studies showing this association.

TABLE 1
Studies showing the association between selected VOCs and bacteria
Selected VOCs and Associated Bacteria

| VOC | Bacteria Identified | Identification Methodology | Reference |
|---|---|---|---|
| Butryic | E. coli, P. mirabili, B. cepacia, S. pyogenes | SIFT-MS, headspace of monoculture | 1 |
| Acid | | after 5 hours incubation (24 for EF) | |
| | Actinomyces naeslundii, Clostridium bifermentans, | Head-space solid phase microextraction | 3 |
| | Clostridium perfringens, Clostridium septicum, | combined with gas chromatography | |
| | Clostridium sporogenes, Clostridium tertium, | cultivated on Vf medium sampled as 1 ml | |
| | Eubacterium lentum, Eubacterium sp., Fusobacterium | | |
| | simiae, Fusobacterium necrophorum, Lactobacillus acidophilus, | | |
| | Peptococcus niger, Peptostreptococcus anaerobius, | | |
| | Peptostreptococcus asaccharolyticus, | | |
| | Peptostreptococcus prevotii | | |
| Hydrogen | P. aeruginosa, S. aureus, E. coli, P mirabilis | SIFT-MS, headspace of monoculture | 1 |
| Sulphide | B. cepacia, E. faecalis | | |
| | S. pneumoniae, E. coli, N. meningitidis | SIFT-MS, blood infected headspace, | 2 |
| | | anaerobic conditions for 24 hours | |
| Pentanol | S. aureus, E. coli, P. mirabili, B. cepaci, | SIFT-MS, headspace of monoculture | 1 |
| | S. pyogenes, E. faecalis | | |
| Propanoic | Acinetobacter baumannii, Actinomyces naeslundii | Head-space solid phase microextraction | 3 |
| Acid | Actinomyces naeslundii, Bacteroides capillosus, | combined with gas chromatography | |
| | Bacteroides fragilis, Bacteroides pyogenes, | cultivated on Vf medium sampled as 1 ml | |
| | Clostridium bifermentans, Clostridium difficile, | | |
| | Clostridium perfringens, Clostridium ramosum | | |
| | Clostridium septicum, Clostridium sporogenes, | | |
| | Clostridium tertium, Escherichia coli, | | |
| | Enterobacter cloacae, Eubacterium lentum, | | |
| | Eubacterium sp., Fusobacterium simiae, | | |
| | Fusobacterium necrophorum, Klebsiella pnuemoniae | | |
| | Lactobacillus acidophilus, Nocardia sp. | | |
| | Peptostreptococcus anaerobius, Peptostreptococcus prevotii | | |
| | Propionibacterium acnes, Propionibacterium propionicum | | |

The following provides examples of bacteria whose presence can be indicated by the detection of signature compounds, and examples of the substrates that can be fed to the bacteria to increase the concentration of the signature compounds.

Clostridium spp. can be detected by initially feeding the bacteria with substrate compounds, acetic acid and/or ethanol, which are metabolised into signature compounds which are detectable. Excess acetic acid produces butyric acid, and excess ethanol produces caproic acid. These signature compounds can be measured to thereby detect the presence of Clostridium spp.

Lactobacillus spp. can be similarly detected by feeding it first with the substrate, lactic acid, which is converted into acetic acid, 1,2-propanediol, and ethanol. These signature compounds can be measured to detect the presence of Lactobacillus spp.

Lactobacillus, Clostridia and other bacteria can be detected by feeding with glutamate. Glutamate is converted to gamma amino butyric acid. This signature compound can be measured to detect the presence of Lactococcus spp., Clostridium spp., and others.

Klebsiella spp. can be detected by feeding with glycerol. Glycerol is metabolised to 3-hydroxypropionic acid, and this signature compound can be measured to detect the presence of Klebsiella spp.

### Example 2

### An Augmented Microbiome-Mediated Breath Test for the Earlier Diagnosis of Oesophago-Gastric Cancer (AMBEC)

The inventors have developed a non-invasive test for oesophago-gastric adenocarcinoma (specificity 81%/sensitivity 80%) based on the detection of volatile organic compounds (VOCs) in exhaled breath. The inventors have improved the accuracy of this test by means of an oral drink which induces the cancer-associated microbiome to produce greater quantities of the distinctive VOCs and thereby allow patients with non-specific symptoms, yet at a high-risk of oesophago-gastric cancer, to be referred faster and earlier for treatment. The oral drink (oral stimulant drink-OSD) selectively ‘feeds’ the cancer-associated microbiome with substances that it will preferentially metabolise to generate quantifiably higher levels of distinctive VOCs. Briefly, the patient is fasted for at least 4 hours and then, while at rest for 20 minutes, breathes into a bag or using breath collection device (such as ReCIVA—see below) that concentrate the volatile compounds into a thermal desorption tube. Breath samples will be analysed.

The test of the invention could be offered immediately by a medical professional in a similar manner to a routine blood test, thus avoiding the need to “watch-and-wait” to see if a patient's symptoms worsen.

The test is intended to be performed by a medical professional, who would then send breath samples to a laboratory for analysis. A positive result would warrant immediate referral for endoscopy. A negative result would permit the medical professional to reassure the patient and offer retesting if symptoms persist.

One example of the invention is referred to as AMBEC (an Augmented Microbiome-mediated Breath Test for the Earlier Diagnosis of Oesophago-gastric Cancer). The target population for testing with AMBEC is patients with upper gastrointestinal symptoms attending GP practices. AMBEC is a highly patient-friendly non-invasive test that will enable both earlier and faster diagnosis, and will substantially mitigate rising pressures on central diagnostic endoscopy.

Patients are given an oral drink (oral stimulant drink—OSD) to stimulate VOC production by the oesophago-gastric cancer-associated microbiome. The OSD is administered and a breath test is undertaken at 30 minute-intervals for 2 hours.

Breath is collected by a low-cost device and analysed in regional laboratories using automated standard mass-spectrometry equipment, such as the apparatus shown in FIG. 1.

Referring to FIG. 1, there is shown an ReCIVA apparatus used for the breath sampling in accordance with the invention. The ReCIVA apparatus is a reproducible system that allows direct breath collected into the thermal desorption tubes, which is the system to be used in future multi-centre studies.

Breath was collected using 500 ml inert aluminium bags that were washed through with synthetic air prior to sampling. Patients were asked to perform deep nasal inhalation followed by complete exhalation through the mouth into secure GastroCHECK steel breath bag. Alveolar air was preferentially collected over dead space air by capturing end-expiratory breath. VOCs from breath bags were then pre-concentrated (see Figure i) onto thermal desorption tubes by transferring 250 ml of breath at 50 ml sec across the tubes with comm diameter tubing and hand-held air pumps (210-1002 MTX, SKC ltd., Dorset, UK).

Patients are fasted for a minimum of four hours prior to breath sample collection. All breath samples are collected prior to endoscopy or surgery.

Exhaled breath analysis can be performed using GC-MS as the standard identification technique, and PTR-TOF-MS as the quantitative technique with a Time-of-Flight analyser to guarantee cutting-edge performance in terms of mass and time resolution.

### Example 3

### Studies Showing the Association between Cancers and Bacteria

The studies shown in Table 2 indicate bacteria that have been shown to be associated with particular cancer types. Hence, enabling the diagnosis of these cancers by the detection of these bacteria, for example in a patient's microbiome.

TABLE 2
Bacteria associated with cancer

| Author and year | Analytical | Cancer | Cancer | Cancer | Healthy | |
|---|---|---|---|---|---|---|
| of Publication | Platform | site | type | (N) | (N) | Bacteria |
| Johan Dicksved | 16sRNA | Gastric | GC | 6 | 15 | Streptococcus |
| Microbiology | | | | | | Lactobacillus |
| 2009, DOI: | | | | | | Veillonella |
| 10.1099/ | | | | | | Prevotella |
| jmm.0.007302-0. | | | | | | Neisseria |
| | | | | | | Haemophilus |
| Francisco | 16sRNA | Gastric | GC | 5 | 5 | Streptococcus |
| Scientific Reports | | | | | | Lactobacillus |
| 2014, DOI: | | | | | | Veillonella |
| 10.1038/srep04202. | | | | | | L. coleohomini |
| | | | | | | Lachnospiraceae |
| Chang Soo Eun | 16sRNA | Gastric | GC | 11 | 10 | Streptococcus |
| Helicobacter | | | | | | Lactobacillus |
| 2014, DOI: | | | | | | Veillonella |
| 10.1111/hel.12145. | | | | | | Prevotella |
| Yalda | 16sRNA | Gastric | GC | 8 | 185 | Streptococci |
| Scientific World | | | | | | Lactobacilli |
| Journal Volume | | | | | | Neisseria |
| 2014, DOI: 1421. | | | | | | Klebsiella |
| Nasrollahzadeh | 16sRNA | Esophagus | ESCC | 37 | 17 | Clostridiales |
| Scientific Reports | | | | | | Erysipelotrichales |
| 2015, DOI: | | | | | | |
| 10.1038/srep08820. | | | | | | |

### Example 3

### Development of Augmented Microbiome-Mediated Breath Test for the Earlier Diagnosis of Oesophago-Gastric Cancer (AMBEC)

(i) Production of an Oral Stimulant Drink (OSD)

The aim was to develop an enhanced OSD formulation that enables the inventors to fully optimise the dose-response, reproducibility and robustness of the new triage test for clinical introduction. The production of the OSD was based on: (i) the dataset of gastric microbiomal bacteria most commonly associated with cancer tissue, (ii) an extensive bioinformatics review of the enzymatic pathway regulation and biochemical io flux in key bacterial species, (iii) the scientific literature describing the conversion of particular primary metabolites to specific VOCs, and (iv) ethical, safety and acceptability considerations of OSD components, such as normal dietary presence, recommended daily allowance (RDA) and palatability.

Sugars, organic acids and amino acids were identified as priority compounds.

Accordingly, specific stimuli were selected for the initial OSD. Several fatty acid stimuli were discounted due to insolubility within the aqueous OSD formulation. A suitable commercial kitchen was identified for OSD manufacture. All OSD components and consumables were sourced as either “food” or “medical” grade to ensure no possibility of contamination and that the drink is fit for human consumption.

Results: Table 3 summarises the composition of one embodiment of the OSD.

TABLE 3
OSD composition

| | Stimulant Mix | | RDI |
|---|---|---|---|
| | mg/mL_{water} | mg/kg_{bodyweight}* | mg/kg_{bodyweight}/d |
| Tyrosine | 1.75 | 2.5 | 25 |
| Glutamic Acid | 21.0 | 30 | 30 |
| Glucose | 130.2 | 186 | 1857 ** |
| Sorbitol | 35.0 | 50 | |
| Lactose | 130.2 | 186 | |
| Glycerol | 193.2*** | 276 | |
| Citric Acid | 14 | 20 | |
| Acetic Acid | 7 | 10 | |

The OSD was prepared under ISO9001 principles of Quality Management with full traceability of batch records. The feasibility results from the OSD provide the proposed further work with an excellent basis for the critical intervention necessary to elicit an augmented response.
RDI—recommended daily intake
*Total bodyweight defined here as 70 kg.
** Estimated for a 70 kg healthy adult.
***~29% lower than glycerol content in Covonia cough syrup.

In one embodiment, the OSD is in the form of a capsule that is designed to degrade at a certain position with the gastrointestinal tract, thereby offering targeted release of the at least one substrate. In another embodiment, the OSD is a liquid drink. Glucose, lactose and sorbitol are believed to be most important for augmenting the microbiome to produce the signature VOCs.

(ii) VOC Production by Microbiome Associated with Patient Cancer Types

The aim was to identify dominant microbiome (bacterial species) associated with oesophago-gastric cancer. Dominant microbiomes associated with oesophago-gastric cancer were identified from: (i) a literature search, (ii) 16S analysis of cancer and normal tissue samples, and (iii) microbiome cultures from oesophageal and gastric cancer and non-cancer tissues obtained from oesophagio-gastric cancer and control patients.

(A) 16S Analysis of Cancer and Normal Tissue Samples Methods: 16S RNA sequencing analysis was undertaken upon gastric and oesophago-gastric tissue samples obtained during surgery. Samples were subjected to metataxonomic analysis on the Illumina MiSeq platform, with the V₃/V₄ region of OG cancer microbiomes being targeted in a high-multiplexing approach, thus leading to a high coverage of the microbial diversity. Taxonomic-dependent analysis of reads from amplicon sequencing was performed using Mothur software. Comparison of dominant bacterial phyla within cancer and non-cancer samples using univariate statistical analysis was performed. Supervised and unsupervised statistical modelling was performed with incorporation of clinical metadata. Results: The inventors have identified the presence of a number of bacteria associated with cancer, as shown in Table 4.

TABLE 4
Elevated VOC levels in active biotrans verses controls

| | Glycerol media | Glucose media |
|---|---|---|
| E. coli | Acetate, propionoic acid, | Hexanal, butanoic acid, |
| (NCIMB 9552) | butanoic acid | pentanoic acid, hexanoic |
| | | acid |
| L. fermentum | Acetaldehyde, Heptanal, | Acetone |
| (NCIMB 11840) | ethyl phenol | |
| S. Salivarius | Acetate, pentanoic acid, | Insufficient growth |
| (NCIMB 701779) | octanal | |
| S. Anginosus | Acetone, Butnaoic acid, | Butanoic acid, Hexanal |
| (NCIMB 702496) | Hexanal, Ethyl phenol, | |
| | Nonanal | |
| K. pneumoniae | Hexanoic acid | Butanoic acid, pentanoic |
| (NCIMB 13281) | | acid |

(Stimuli cocktail composition, all at 0.1 M concentration: tyrosine, glutamic acid, glucose, lactose, sorbitol, glycerol, ethanol, xylose, phenylalanine)

As shown in Table ₄, higher abundance of Firmicutes (Lactobacillus fermentum, Streptococcus salivarius, Streptococcus anginosus, Klebsiella pneumoniae, Escherichia coli) was found in oesophago-gastric cancer tissue compared to control samples. The identification of dominant oesophago-gastric cancer-associated microbiomes provides target microbiomes to be stimulated by OSD in order to elicit an optimal augmented response. (B) Microbiome Culture from Patient Cancer and Normal Tissue Samples

The aim was to illustrate a difference in VOCs originate from bacteria associated with either cancer or normal tissues obtained from patients in order to provide support for the overall hypothesis that the gastric microbiome can produce markers of cancer presence.

Methods: Frozen samples of cancer tissue and non-cancer control tissue in glycerol-freeze media were used for Sequencing (16S/Shotgun) and headspace analysis. Tissue samples were defrosted and re-suspended in 100 μL PBS (sterile pH ₅) and vortexed vigorously for 60 seconds. 100 μL of supernatant fluid was then spread on pre-prepared FAA (Fastidious Anaerobe Agar+7% Horse Blood) medium on petri plates and incubated in anaerobic ES-Gas pouches at 37° C. for 24 hours. The following day, the plates were removed from the incubator and treated in two ways: (A) Predominant species: Individual bacterial colonies most frequently occurring on each FAA plate were picked and re-suspended in 100 μL PBS in vials for sequencing and headspace analysis; and (B) Overall species composition: 1.5 ml PBS was used to re-suspended all bacteria from individual plates. The resuspension was then pipetted into an eppend.orf tube and micro-centrifuged at 14800 for 5 mins. Supernatant fluid was removed and the solid pellet re-suspended in 100 μL PBS and split into vials for sequencing and headspace analysis via solid phase micro-extraction (SPME-GC-MS) using a carboxen/polydimethysiloxane SPME fibre. SPME extraction was performed at 60° C. with intermittent agitation at 500 μm. Volatiles were collected in the absence of airflow, after 48 hours of incubation followed by direct release into a heated gas chromatography injector.

Results: Differing abundances of volatile aldehydes and fatty acids were detected in the headspace from predominant and total culturable bacterial species associated with cancer and non-cancer samples. For the predominant bacteria (A), VOCs including benzaldehyde and methyl phenol were significantly higher in samples from cancer tissue versus normal tissue controls. As for the overall bacteria present in the tissue samples (B), acetic and butanoic acid were significantly higher in cancer tissue versus normal controls (see FIG. 2).

(iii) In vitro VOC Production by different Microbiomes in response to OSD

A) Standard Bacterial Culture and VOC production in response to RDA of OSD components

The aim was to examine the feasibility to culture relevant microbiomes and analyse their VOC produced in response to stimuli used in the OSD, at human recommended daily allowance concentrations.

Methods: Using the above preliminary clinical data provided by VODCA, and literature references describing microbiome-associations with gastric cancer, suitable strains were obtained from culture collections such as NCIMB (Aberdeen) and ATCC (USA).

See Table 4. All in-vitro culture work was performed under conditions as closely simulating the natural gastric environment as possible (e.g. anaerobiosis, pH_{5.5}) in Cati or Cate laboratories as appropriate, according to UK microbiological regulatory guidelines using well-established Ingenza protocols. All associated quality control testing was performed throughout. Several study parameters were optimised during the work to enhance cell growth, culture sampling and VOC analysis.

Results: E. coli culture grew satisfactorily and generated detectable VOCs, but all other cultures either did not achieve satisfactory growth under the initial protocol or did not produce detectable VOCs at the concentrations of stimuli used.

B) High throughput analysis of VOC production by different human microbiome bacteria

The aims were to: (i) develop a high throughput system that maximises microbiome culture and VOC production and analysis, and (ii) examine VOCs produced by different known cancer-associated microbiome members.

The inventors set out to develop a high-throughput system as the platform for efficient testing of microbiome responses to different OSD compositions and concentrations, to inform the design of subsequent patient dosage studies, recognising that many foods and common nutritional supplements (e.g. vitamins, minerals, amino acids) often greatly exceed the RDAs for compounds potentially suitable in the OSD.

Reasons for revising the initial culture protocol: (i) insufficient microbiome growth under initial protocol in experiment (iii)A, (ii) insufficient VOC levels in response to RDA of OSD compounds in experiment (iii)A, (iii) difficulties found with the use of the OmniLog including slow growth rate of many cancer associated bacteria, and inability to maintain efficient vessel sealing during headspace sampling of highly volatile compounds, and (iv) the inventors' VOC analytical capabilities proving significantly more sensitive than Ingenza's equipment. It was therefore decided to use growth media and conditions that allowed greater culture biomass and increased concentrations of potential VOC stimuli, since in vitro work is not bound by patient safety constraints.

Methods:

Growth media: The mechanisms of genetic and biochemical regulation of the gastric microbes under evaluation was considered important in deciding the composition of laboratory growth media and carbon source used to generate biomass. Glucose mediated catabolite repression can inhibit enzymes necessary to catabolise alternate carbon sources. Media rich in supplements such as amino acids or metabolic intermediates also represses bacterial biosynthesis of these compounds by enzymes that are non-essential under these conditions but whose activity may be required for VOC production. Defined minimal salts medium lacking non-essential supplementation was therefore used.

Stimuli: Concentrations of stimulant constituents were significantly increased, permitting much broader assessment of individual stimulant thresholds, temporal profiles and concerted effects of stimuli upon microbial VOC production. Microbiomes: 5 prioritised bacterial species (Lactobacillus fermentum, Streptococcus salivarius, Streptococcus anginosus, Klebseilla pneumonia, E. coli) were cultured with glucose or glycerol carbon sources.

Procedure: A protocol was established for biomass generation in shake flask cultures followed by VOC stimulation in 50 ml falcon tubes. Final nitrogen sparging and high-throughput VOC sampling in headspace vials provided the required accuracy and reproducibility with no loss of throughput (see FIG. 3). VOC capture and transport: Headspace VOCs were captured on conditioned thermal desorption (TD) tubes and shipped in ice-packs to Imperial in batches of 50-100 tubes. TD tubes allow stable storage and transport of target VOCs for 72 hours at room temperature and 4 weeks at −20° C.

VOC analysis: Analysis was conducted at St Mary's VOC laboratory using standard Gas chromatography mass-spectrometry (TD/GC-MS) and Proton transfer reaction time-of-flight mass-spectrometry (TD/PTR-TOF-MS).

Results: Samples were collected for all cultures and specific VOCs found to be elevated 2-10 fold over controls in particular cultures (see Table 4 and FIG. 4). The data indicated specific bacterial fatty acid, phenol and aldehyde VOCs were produced in response to the stimuli included in the culture media. Key elevated VOCs were pentanoic acid, hexanoic acid, butyric acid, acetic acid, acetaldehyde, hexanal, octanal, heptanal, phenol and ethyl phenol.

(iv) Clinical Study

Ethical approval: REC Reference (18/LO/0078).

Hypothesis: Cancer cells and their associated bacteria will be able to utilise administered substrates within defined metabolic pathways responsible for the production of VOC's. By exploiting inherent metabolic pathways in this way we expect to observe a transient elevation in cancer associated VOCs.

Methods: Patients with cancer of the oesophageal or stomach as well as subjects with a normal upper gastrointestinal tract were recruited at the time of routine outpatient assessment. Patients were required to fast overnight prior to breath sampling. A baseline breath sample was collected at the start of the study period by asking participants to exhale directly into a double thickness Nalophan® bag (Kalle UK Ltd, UK). Participants were then asked to consume the OSD. Following consumption of the OSD, participants were asked to rinse their mouth with water in order to eliminate any oral residue of the OSD. Further breath samples were then collected at 30 and 60 minutes following ingestion of the OSD. Breath samples were transferred from Nalophan® bags on to thermal desorption tubes using a precision handheld pump (SKC Ltd, UK).

VOC analysis: Breath samples were analysed by PTR-TOF-MS and GC-MS techniques for target quantification of cancer biomarkers: fatty acids (acetic acid, butyric acid, pentanoic acid and hexanoic acid) phenol, ethyl phenol and aldehydes. Exhaled acetone, a marker of ketosis (a state of energy depletion) was assessed in order to verify the administration of a nutritional stimulus. Strict quality control measures were followed.

Results: 30 patients with gastroesophageal cancer and 30 control subjects were recruited. All participants were able to consume the OSD and there were no observed or reported adverse events. Acetone levels in both cancer and control subjects decreased following ingestion of the OSD confirming nutritional stimulation that occurred. Following ingestion of the OSD target VOCs in cancer patients (pentanoic acid, hexanoic acid, butyric acid, acetic acid, phenol, ethyl phenol) were detected at higher levels as indicated by the average fold change in VOC concentrations at 30 and 60 mins. With the exception of butyric acid (30 mins time point), control subjects exhibited a ≤10% variation in target VOC levels following ingestion of the OSD. Mean fold change variation in exhaled hexanoic acid, and pentanoic acid is presented in (see Table 5 and FIG. 4). For cancer patients who had previously received chemoradiotherapy OSD, response for pentanoic acid appeared to be suppressed such that it was similar to control subjects.

TABLE 5
Mean fold change in select exhaled VOCs following
administration of oral stimulant drink

| | Cancer | | | Controls | | |
|---|---|---|---|---|---|---|
| | 0 mins | 30 mins | 60 mins | 0 mins | 30 mins | 60 mins |
| Acetone | 1.0 | 1.0 | 0.9 | 1.0 | 1.0 | 0.9 |
| Acetic Acid | 1.0 | 1.1 | 1.2 | 1.0 | 1.0 | 0.9 |
| Butyric Acid | 1.0 | 1.6 | 1.3 | 1.0 | 1.2 | 1.0 |
| Pentanoic Acid | 1.0 | 1.1 | 1.2 | 1.0 | 0.9 | 1.0 |
| Hexanoic Acid | 1.0 | 1.1 | 1.2 | 1.0 | 1.0 | 1.0 |
| Phenol | 1.0 | 1.2 | 1.3 | 1.0 | 1.0 | 0.9 |
| Ethyl Phenol | 1.0 | 1.2 | 1.3 | 1.0 | 0.9 | 1.0 |
| Acetaldehyde | 1.0 | 1.1 | 1.2 | 1.0 | 1.1 | 1.1 |

Data is derived from breath samples analysed by PTR-TOF-MS (OSD composition at the concentrations listed in Table 3: tyrosine, glutamic acid, glucose, lactose, sorbitol, glycerol, citric acid, acetic acid.

TABLE 6
Columns 2-5: Mean differences of the log-transformed data between cancer patients
and controls and pooled variances at 30 and 60 minutes. Columns 6-7: Sample size
calculation and power calculation in all compounds for the chosen sample size

| | Mean diff | Mean diff | | | | |
|---|---|---|---|---|---|---|
| | (cases − | (cases − | Pooled | Pooled | Final | Power |
| | controls) | controls) | variance | variance | sample size | with |
| Compound | time = 30 min | time = 60 | time = 30 | time = 60 | (Power 90%) | n = 188 |
| Acetic Acid | 0.069 | 0.155 | 0.155 | 0.205 | 188 | 90% |
| Butyric Acid | 0.138 | 0.260 | 0.310 | 0.249 | 81 | 99.7% |
| Phenol | 0.050 | 0.232 | 0.329 | 0.420 | 171 | 92.2% |
| Pentanoic Acid | 0.139 | 0.106 | 0.164 | 0.221 | 186 | 90.2% |
| Hexanoic Acid | 0.176 | 0.203 | 0.221 | 0.168 | 90 | 99.5% |
| Ethyl Phenol | 0.219 | 0.205 | 0.113 | 0.230 | 52 | 100.0% |

The final sample size was chosen as the minimum sample size between both time points based on the expected maximum difference between cases and controls. The inventors used the formula (3·31) in Chapter 3 (Julious, S A. Sample sizes for clinical trials. 2010-Chapman and Hall) for comparison of two means in a parallel study adjusting for the imprecision of the population variance estimation and assuming the same number of cases and controls.

VOCs belonging to fatty acids, phenols and aldehydes were produced by cancer-associated microbiomes cultured from commercial strains and cancer tissues obtained from patients with oesophago-gastric adenocarcinoma. Different microbiomes produced distinct VOC profiles. As shown in Tables 5 and 6, key elevated VOCs include pentanoic acid, hexanoic acid, butyric acid, acetic acid, butanoic acid, acetaldehyde, benzaldehyde, hexanal, octanal, heptanal, phenol, methyl phenol and ethyl phenol.

Conclusions: Preliminary investigations have demonstrated ‘proof or principle’ that exhaled biomarkers of gastroesophageal cancer could be augmented by an OSD. The findings from in-vitro bacterial culture experiments provide evidence that the cancer associated bacteria are, at least in part, responsible for the observed changes in target VOC. The findings have also indicated that this response may be suppressed by the prior chemoradiotherapy, which is known to modify the intestinal microbiome.

Knowledge of the VOC profile produced by different cancer-associated microbiomes can then be used to:
Generate VOC calibration curves for GC-MS qualitative analysis,
Test the response of microbiomes to different combinations and concentrations of OSD components in a high-throughput microbiome culture system.

### SUMMARY

The inventors have unequivocally demonstrated an increase in the generation of VOCs in patients with oesophago-gastric cancer in comparison to non-cancer subjects in response to the oral stimulant drink (OSD). A major finding has been to obtain common stimulus-inducible VOCs in both the clinical study and in vitro microbiome culture of known cancer-associated bacteria. The inventors, therefore, have a very high confidence in the results because of the consistency of VOC identification using multiple analytical platforms (i.e. GC-MS and PTR-TOF-MS).

In addition, VOCs discovered in AMBEC are among volatile biomarkers that were found to differentiate oesophago-gastric cancer patients from control patients in the initial non-augmented breath test clinical studies (Ann Surg. 2015 Dec; 262(6):981-90. JAMA Oncol. 2018 May 17). These findings provide the basis for further work with the primary objective of establishing an AMBEC protocol that achieves a higher diagnostic accuracy than the 85% shown in previous non-augmented breath analysis studies.

The novelty of the work is using the cancer-associated microbiome to elicit a diagnostic augmented VOC response. In order to realise this novelty, the inventors have achieved:
Production of an oral drink that stimulates the cancer associated-microbiome.
In-vitro demonstration of VOC production in the headspace of microbiome derived cultures in response to OSD as a means to achieve the most significant cancer dependent response in vivo.
Conducting of a clinical trial that showed an increase in the generation of VOCs in patients with oesophago-gastric cancer in comparison to non-cancer subjects in response to OSD.
Development of techniques and detailed protocols for a high throughput system for optimum culturing and testing of the cancer-associated microbiome and capturing generated VOCs, as well as VOC transport and analysis. This high throughput system is transferable to other cancers to explore microbiome augmented diagnostic responses.

### REFERENCES

1. Thorn R., Reynolds D., Greenman J., Multivariate analysis of bacterial volatile compound profiles for discrimination between selected species and strains in vitro. Journal of Microbiological Methods. 2011; 84(2): 258-264.

2. Allardyce R., Hill A., Murdoch D., The rapid evaluation of bacterial growth and antibiotic susceptibility in blood cultures by selected ion flow tube mass spectrometry. Diagnostic Microbiology and Infectious Disease. 2006; 55(4): 255-261.

3. Julak J., Prochazkova-Francisci E., Stranka E., Rosova., Evalutation of exudates by solid phase microextraction-gas chromatography. Journal of Microbiological Methods. 2003; 52(1): 115-122.

4. Altomare D F, Di Lena M, Porcelli F, et al. Exhaled volatile organic compounds identify patients with colorectal cancer. Br J Surg 2013; 100: 144-150

5. Spanel P, Smith D. Selected ion flow tube mass spectrometry for on-line trace gas analysis in biology and medicine. Eur J Mass Spectrom 2007; 13: 77-82 12. Spanel P, Smith D, Progress in SIFT-MS: breath analysis and other applications. Mass Spectrom Rev 2011; 30: 236-267

6. Altomare D F, Di Lena M, Porcelli F, et al. Effects of curative colorectal cancer surgery on exhaled volatile organic compounds and potential implications in clinical follow-up. Ann Surg 2015; 262: 862-866.

1. A method for treating a subject suffering from cancer, the method comprising:
(i) detecting, in a bodily sample from a test subject, the concentration of a signature compound resulting from the metabolism, by a cancer-associated microorganism, of at least one substrate in a composition previously administered to the subject; and
(ii) comparing the concentration of the signature compound with a reference for the concentration of the signature compound in an individual who does not suffer from the cancer, wherein an increase or a decrease in the concentration of the signature compound compared to the reference, indicates that the subject is suffering from the cancer; and
(iii) administering a therapeutic agent capable of treating the cancer to the test subject whose concentration of the signature compound in the bodily sample indicates that the subject is suffering from the cancer.

2. The method according to claim 1, further comprising providing the subject with the composition comprising the at least one substrate which is suitable for metabolism by the cancer-associated microorganism into the signature compound.

3. (canceled)

4. The method according to claim 1, wherein the cancer-associated microorganism is associated with oesophago-gastric junction cancer, gastric cancer, oesophageal cancer, oesophageal squamous-cell carcinoma (ESCC), or oesophageal adenocarcinoma (EAC), and wherein the cancer is oesophago-gastric junction cancer, gastric cancer, oesophageal cancer, oesophageal squamous-cell carcinoma, or oesophageal adenocarcinoma.

5. (canceled)

6. The method according to claim 1, wherein the composition comprising the at least one substrate, which is suitable for metabolism by the cancer-associated microorganism into the signature compound, is ingestable by the subject.

7. The method according to claim 1, wherein the composition comprising the at least one substrate is: (i) in the form of a capsule that is designed to degrade at a certain position with the gastrointestinal tract, thereby offering targeted release of the at least one substrate; or (ii) is a solid, foodstuff, fluid or liquid, which is swallowed.

8. The method according to claim 1, wherein the at least one substrate is selected from a group consisting of: acetic acid, ethanol, lactic acid, lactate, glutamate, glycerol, D-glucose, D-sucrose, D-lactose, D-fructose, D-mannose, D-gulose, D-galactose, D-Xylose, D-arabinose, D-lyxose, D-ribose, L-arabinose, L-rhamnose, L-xylulose, di-, tri-oligo and poly-saccharides, c4, c7 and >c8 monosaccharides, pyruvic acid, ascorbic acid, malic acid, citric acid, succinic acid, fumaric acid, oxalic acid, tannic acid, tartaric acid, sorbitol, mannitol, maltitol, lactitol, erythritol, palmitic acid, stearic acid, oleic acid, linoleic acid, arachidonic acid, triglycerides, glycolipids, any or all of the 20 proteinogenic amino acids, 2-amino butyric acid, ornithine, canavanine, homoarginine, artificial sweeteners, stevia, aspartame, sucralose, preservatives, E numbers, nitrate, and small molecule inducers.

9. The method according to claim 1, wherein the composition comprises one or more substrates selected from the group consisting of: glucose, sorbitol, lactose tyrosine, glutamic acid, glycerol, citric acid and acetic acid, or any combination thereof.

10. The method according to claim 1, wherein the cancer-associated microorganism is a bacterium.

11. The method according to claim 1, wherein the cancer-associated microorganism forms part of the microbiome of the test subject.

12. The method according to claim 1, wherein the cancer-associated microorganism is Streptococcus, Lactobacillus, Veillonella, Prevotella, Neisseria, Haemophilus, L. coleohominis, Lachnospiraceae, Klebsiella, Clostridiales, Erysipelotrichales, or any combination thereof.

13. The method according to claim 1, wherein the cancer-associated microorganism is S. pyogenes, Klebsiella pneumoniae, Lactobacillus acidophilus, or any combination thereof.

14. The method according to claim 1, wherein the cancer-associated microorganism is E. coli, P. mirabili, B. cepacia, Streptococcus salivarius, Streptococcus anginosus, S. pyogenes, Actinomyces naeslundii, Lactobacillus fermentum, Clostridium bifermentans, Clostridium perfringens, Clostridium septicum, Clostridium sporogenes, Clostridium tertium, Eubacterium lentum, Eubacterium sp., Fusobacterium simiae, Fusobacterium necrophorum, Lactobacillus acidophilus, Peptococcus niger, Peptostreptococcus anaerobius, Peptostreptococcus asaccharolyticus, Peptostreptococcus prevotii, P. aeruginosa, S. aureus, P. mirabilis, E. faecalis, S. pneumoniae, N. meningitides, Acinetobacter baumannii, Bacteroides capillosus, Bacteroides fragilis, Bacteroides pyogenes, Clostridium difficile, Clostridium ramosum, Enterobacter cloacae, Klebsiella pneumoniae, Nocardia sp., Propionibacterium acnes, Propionibacterium propionicum, or any combination thereof.

15. The method according to claim 1, wherein the signature compound is a volatile organic compound (VOC).

16. The method according to claim 15, wherein the volatile organic compound (VOC) is selected from a group consisting of: butyric acid, gamma amino butyric acid, caproic acid, hydrogen sulphide, pentanol, propanoic acid, acetic acid, 1,2-propanediol, ethanol, and 3-hydroxypropionic acid, or any combination thereof, optionally acetone, acetic acid, butyric acid, pentanoic acid, hexanoic acid, phenol, ethyl phenol, acetaldehyde, or any combination thereof, or hexanoic acid, pentanoic acid, acetic acid, 2 ethyl phenol, or any combination thereof.

17. The method according to claim 1, wherein the VOC is selected from a group consisting of: aldehydes, fatty acids, and alcohols, or any combination thereof.

18. The method according to claim 1, wherein the cancer is oesophageal squamous-cell carcinoma, wherein the composition comprises a substrate selected from acetic acid and/or ethanol, which is metabolised to a signature compound selected from butyric acid and/or caproic acid, optionally which is analysed to indicate the presence of Clostridium spp.

19. The method according to claim 1, wherein the cancer is gastric cancer, wherein the composition comprises a substrate which is lactic acid, which is metabolised to a signature compound selected from acetic acid, 1,2-propanediol, and/or ethanol, optionally which is analysed to indicate the presence of Lactobacillus spp.

20. The method according to claim 1, wherein the cancer is oesophago-gastric cancer, wherein the composition comprises a substrate which is glutamate, which is metabolised to a signature compound which is gamma amino butyric acid, optionally which is analysed to indicate the presence of Lactococcus spp. or Clostridium spp.

21. The method according to claim 1, wherein the cancer is gastric cancer, wherein the composition comprises a substrate which is glycerol, which is metabolised to a signature compound which is 3-hydroxypropionic acid, optionally which is analysed to indicate the presence of Klebsiella spp.

22. (canceled)

23. (canceled)

24. (canceled)

25. The method according to claim 1, wherein the composition comprises:
(i) glucose at a concentration of between about 7000 mg/100 mL and 20000 mg/100 mL, or between about 9000 mg/100 mL and 17000 mg/100 mL, or between about 11000 mg/100 mL and 15000 mg/100 mL;
(ii) lactose at a concentration of between about 7000 mg/100 mL and 20000 mg/100 mL, or between about 9000 mg/100 mL and 17000 mg/100 mL, or between about 11000 mg/100 mL and 15000 mg/100 mL;
(iii) sorbitol at a concentration of between about 1000 mg/100 mL and 6000 mg/100 mL, or between about 2000 mg/100 mL and 5000 mg/100 mL, or between about 3000 mg/100 mL and 4000 mg/100 mL;
(iv) tyrosine at a concentration of between about 25 mg/100 mL and 500 mg/100 mL, or between about 50 mg/100 mL and 400 mg/100 mL, or between about 100 mg/100 mL and 300 mg/100 mL;
(v) glutamic acid at a concentration of between about 500 mg/100 mL and 5000 mg/100 mL, or between about 1000 mg/100 mL and 3500 mg/100 mL, or between about 1500mg/100mL and 2500mg/100mL;
(vi) glycerol at a concentration of between about 10000 mg/100 mL and 30000 mg/100 mL, or between about 14000 mg/100 mL and 25000 mg/100 mL, or between about 17000 mg/100 mL and 22000 mg/100 mL;
(vi) citric acid at a concentration of between about 500 mg/100 mL and 3000 mg/100 mL, or between about 1000 mg/100 mL and 2000 mg/100 mL, or between about 1200 mg/100 mL and 1700 mg/100 mL; and/or
(vii) acetic acid at a concentration of between about 200 mg/100 mL and 1500 mg/100 mL, or between about 400 mg/100 mL and 1000 mg/100 mL, or between about 600 mg/100 mL and 800 mg/100 mL.

26. (canceled)

27. (canceled)
